(19) United States
(12) Patent Application Publication
(10) Pub. No.: US 2008/0103425 A1
(43) Pub. Date: May 1, 2008

(54) Scope sock

(76) Inventor: Gregory C. Berlet, Columbus, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 11/974,687

(22) Filed: Oct. 15, 2007

Related U.S. Application Data
(60) Provisional application No. 60/854,854, filed on Oct. 27, 2006.

(51) Int. Cl. A61F 5/00

(52) U.S. Cl. 602/36; 602/32; 602/35

(57) ABSTRACT

An orthopedic distraction sock for circumscribing at least a portion of an appendage, the distraction sock comprising a hollow braided sleeve having opposing open ends and including a non-braided tether mounted to the sleeve so that tension on the tether constricts the sleeve. The instant invention also includes a method of securing the relative position of an ankle during an arthroscopic procedure, the method comprising: (a) retarding movement of an upper leg; (b) sliding a constrictable sleeve axially over at least a majority of a foot, the sleeve including a distal open end and a proximal open end nearer an ankle than the distal open end; (c) mounting the sleeve to a support structure; and (d) tensioning the sleeve by increasing a distance between the support structure and the foot to constrict the sleeve circumferentially around the foot to securely mount the sleeve to the foot, where tensioning of the sleeve, after retarding movement of the femur, is operative to maintain the relative orientation between the foot and a lower leg.

### CROSS REFERENCE TO RELATED APPLICATIONS

This application claims the benefit of U.S. Provisional Patent Application Ser. No. 60/854,854, filed Oct. 27, 2006, and entitled “SCOPE SOCK,” the disclosure of which is incorporated herein by reference.

### FIELD OF THE INVENTION

The present invention is directed to orthopedic equipment and techniques for using such equipment and, more specifically, to orthopedic equipment for distraction of an appendage and techniques for distracting appendages using the orthopedic equipment of the present invention.

### INTRODUCTION TO THE INVENTION

The instant invention includes an orthopedic distraction sock for circumscribing at least a portion of an appendage, the distraction sock comprising a hollow braided sleeve having opposing open ends and including a non-braided tether mounted to the sleeve so that tension on the tether constricts the sleeve. In a more detailed embodiment, the sleeve includes a plurality of attachment loops distributed generally in parallel with a longitudinal axis of the sleeve for secondarily mounting the sleeve to a support structure. In a further detailed embodiment, the sleeve includes a plurality of attachment loops distributed along a length of the sleeve generally perpendicular to a longitudinal axis of the sleeve for secondarily mounting the sleeve to a support structure. In a still further exemplary detailed embodiment, the sleeve is a bias weaved and a major portion of the sleeve is substantially cylindrical. The exemplary embodiment may also include a repositionable loop mounted to the sleeve, wherein the tether is mounted to a distal open end of the sleeve and the repositionable loop is mounted to a proximal open end of the sleeve. In yet another embodiment, the tether comprises a plurality of lines circumferentially mounted to the sleeve approximate a distal open end of the sleeve and bound to have a substantially common point of origin. In an even further embodiment, the sleeve comprises a plurality of braided layers.

A second exemplary embodiment includes an orthopedic distraction sock for circumscribing at least a portion of an appendage, the distraction sock comprising: (a) a constrictable sleeve having opposing first and second open ends; (b) a tether mounted to the sleeve; and (c) a constrictor entwined with the sleeve approximate the first open end.

A third exemplary embodiment includes a method of securing the relative position of an ankle during an arthroscopic procedure, the method comprising: (a) retarding movement of an upper leg; (b) sliding a constrictable sleeve axially over at least a majority of a foot, the sleeve including a distal open end and a proximal open end nearer an ankle than the distal open end; (c) mounting the sleeve to a support structure; and (d) tensioning the sleeve by increasing a distance between the support structure and the foot to constrict the sleeve circumferentially around the foot to securely mount the sleeve to the foot, where tensioning of the sleeve, after retarding movement of the femur, is operative to maintain the relative orientation between the foot and a lower leg.

### BRIEF DESCRIPTION OF THE DRAWINGS

FIG. 1 is a profile view of an operating table and a distraction assembly of the present invention;

FIG. 2 is a profile view of the operating table and distraction assembly of FIG. 1 in another position;

FIG. 3 is a profile view of the operating table and distraction assembly of FIG. 1 in a further position;

FIG. 4 is a profile view of the operating table and distraction assembly of FIG. 1 in still a further position; and

FIG. 5 is an elevated perspective view of alternate exemplary embodiment in accordance with the present invention.

### DETAILED DESCRIPTION

The exemplary embodiments of the present invention are described and illustrated below to encompass orthopedic distraction equipment and methods for using the same. Of course, it will be apparent to those of ordinary skill in the art that the embodiments discussed below are exemplary in nature and may be reconfigured without departing from the scope and spirit of the present invention. However, for clarity and precision, the exemplary embodiments as discussed below may include optional steps, methods, and features that one of ordinary skill should recognize as not being a requisite to fall within the scope of the present invention.

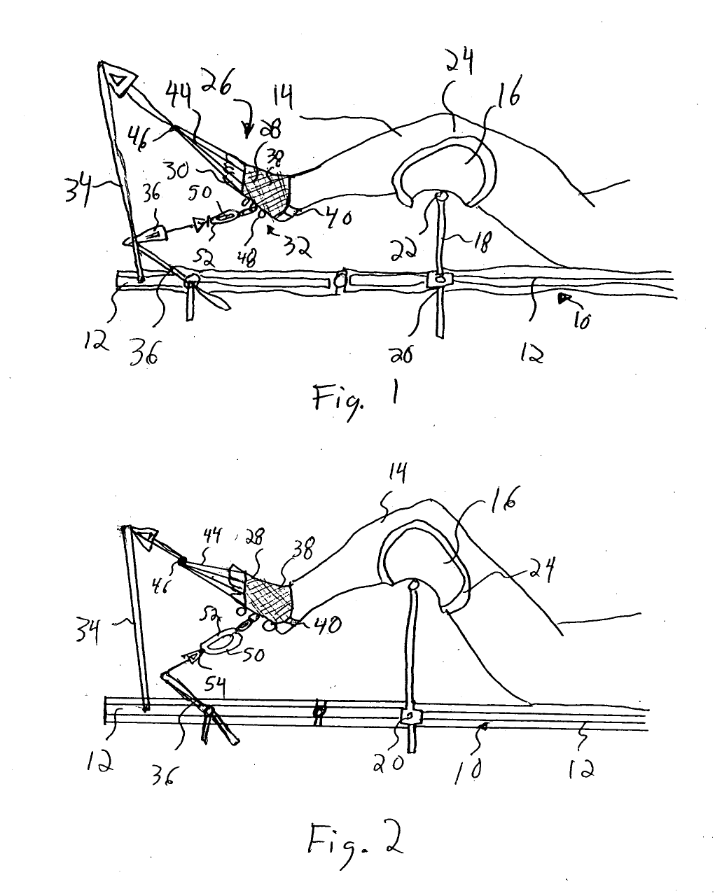

Referring initially to FIG. 1, a conventional operating table 10 includes a standard Clark rail 12 attached to a distal end of the table in a well known manner. The patient is positioned supine on the operating table 10 with the leg 14 of the patient supported at the back of the knee vertically above the table, generally with some flexion of the knee. Leg support is provided by a conventional gynecological or urological leg holder 16 attached in a known manner to the Clark rail 12. The leg holder 16 includes a support shaft 18 adjustably attached to the Clark rail 12 by a clamp 20 that allows the holder to be slid along the rail in a horizontal direction, pivoted in a vertical plane, and moved vertically up or down. The upper end of the support shaft 18 is connected to the leg holder 16 with a pivot joint 22 that allows the holder to be adjustably pivoted in either a horizontal or a vertical plane. The leg holder 16 can easily be adjusted in three planes of rotation and its height readily varied. The leg holder 16 includes padding 24 to provide cushioning to the leg 14, thereby reducing the pressure on the posterior thigh and leg, primarily in the popliteal area.

The distraction apparatus 26 of the present invention includes an axially adjustable braided sock 28 attached proximally to a proximal end 30 of the patient's foot 32 and distally to the operating table 10, via an arm 34, which is adjustably attached to the Clark rail 12. A second arm 36, also attached to the Clark rail 12, is attached to the underside of the sock 28.

Referring to FIGS. 1 and 2, attachment of the sock 28 to the foot 32 of the patient is provided by a braided tubular sleeve 38 having a generally diametrical loop 40 at one axial end, which is placed around and behind the ankle above the heel and instep, and a webbed tether 44 at an opposite axial end to be mounted to the arm 34 by a swivel 46. The sleeve 38 also includes a plurality of rings 48 attached to a circumferential side of the sleeve, such as by interweaving, that are linearly spaced apart and adapted to be oriented parallel to the longitudinal axis of the foot 32 and/or perpendicular to the longitudinal axis of the foot 32 when the sleeve is slid over the foot 32. Attachment of the sleeve 38 to the second arm 36 is facilitated by a tether 50 having a caribiner 52 at one end to engage at least one of the rings 48. The opposite end of the tether 50 is mounted to the second arm 36 by a swivel 54.

Referencing FIG. 2, the tubular sleeve 38 of the sock 28 is braided, such as by using a bias weaving process. This process produces two sets of continuous yarns, one clockwise and the other counterclockwise, where each fiber from one set is interwoven with every fiber from the other set in a continuous spiral pattern. This braided fiber architecture resembles a hybrid of filament winding and weaving. Like filament winding, tubular braid features seamless fiber continuity that is mechanically interlocked with one another. These interlocking connections allow the sleeve to exhibit highly efficient distributions of loads. In terms of compression fitting the sleeve 38 to the contours of the foot 32, the interlocking of the fibers constricts to map the contours and evenly distribute compressive forces about the foot. In particular, braiding of the sleeve 38 offers natural conformability. This quality makes braid ideal for performance as a scope sock sleeve since it takes on the exact shape of the body part that it is mounted to. Therefore, extensive constraints on the sleeve itself are not necessary to mount the sleeve to the body part.

Repeated use of the sock 28 is brought about, in part, by the construction of the sleeve 38, which has excellent resistance to fatigue. Like a filament wound structure, braided fibers are coiled into a helix just like wire in a spring. The difference, however, is the mechanical interlocking. As a structure is exposed to high fatigue cycles, cracks will propagate through the matrix of filament. However, a braided structure arrests the propagation of cracks by intersection of the fibers.

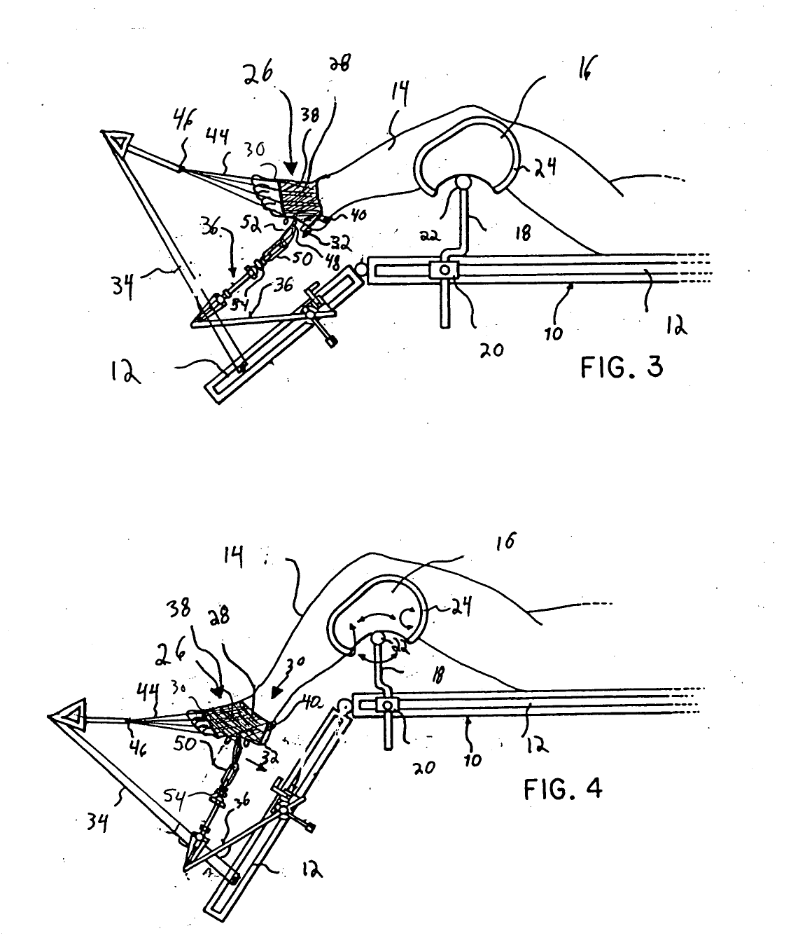

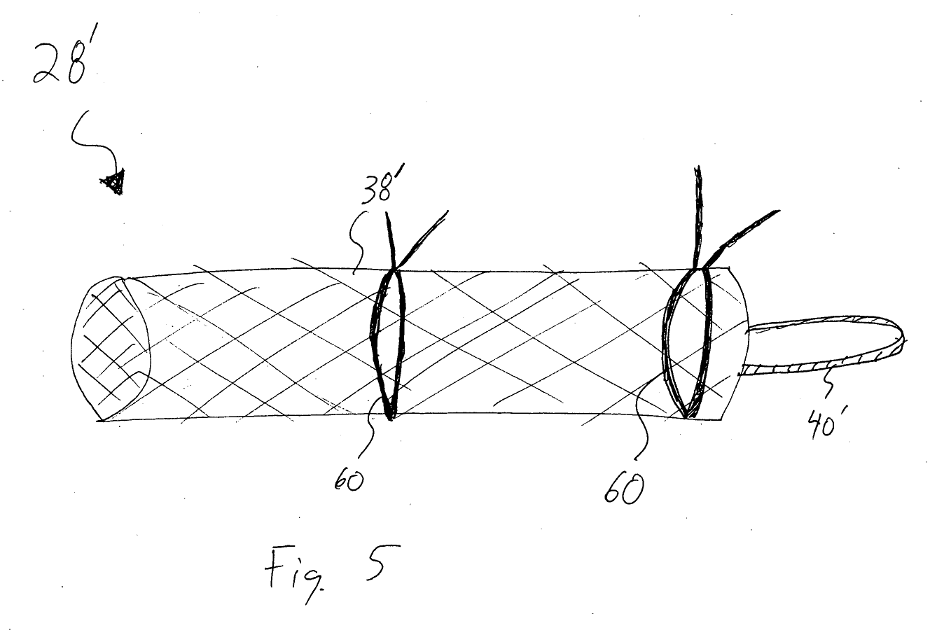

Referring also to FIGS. 3 and 4, the arms 34, 36 of the distraction apparatus 26 each include a threaded bolt to which the swivel 46 is attached. A length adjustment mechanism is attached to the second end of the threaded bolt. The adjustment mechanism includes a manually operable, threaded adjustment nut and an attached mount for an attachment strap. The mount is connected to the adjustment nut with a swivel connection so that the adjustment nut can be rotated on the threaded bolt to change the position of the adjustment mechanism therealong without rotating the sock 28.

The other leg of the lever arm has its free end attached to the Clark rail 12 for adjustable rotational positioning about its free end and axially along the leg. Attachment of the leg to the Clark rail 12 is provided by an adjustable clamp. The clamp includes a clamp body mounted for slidable movement along the Clark rail and a clamp head rotatably attached to the clamp body and movable into clamping engagement therewith by rotation of an operating arm. The clamp body includes a set screw for fixing the position of the clamp along the Clark rail. The clamp head includes a diametral through bore for receiving the leg of the lever arm, and the clamp head is configured internally to simultaneously clamp the leg in the bore and the clamp head to the clamp body by rotation of the operating arm. Similarly, rotation of the operating arm in the opposite direction simultaneously unclamps the clamp head from the body so the head and lever arm may be rotated with respect thereto and the leg can be positioned axially in the through bore.

Arthroscopic procedures utilizing non-invasive distraction techniques are preferably commenced utilizing the distraction position shown in FIG. 1. The support arm for the leg holder is positioned in a mid-height position and fixed with the clamp. With the patient in a supine position, the leg is placed in the leg holder with the knee slightly flexed to about 20 degrees. This initial position of near extension reduces the pressure on the posterior thigh and leg, primarily the popliteal area. The proximal end of the sleeve 38 is placed around the foot and is secured/anchored to the back of the foot by the connecting strap 40. One arm 36 of the distraction apparatus 26 is attached to the underside of the sleeve 38 via one or more of the rings 48, while the distal end of the sleeve 38 is mounted to the arm 34 operative to pull the foot along its longitudinal axis. The clamp bodies are positioned in an appropriate position on the Clark rail and clamped thereto with the set screw. Each arm 34, 36 may then be manually rotated (e.g. by the surgeon's assistant) around the end held in the clamp in a counterclockwise direction as viewed in FIG. 1. The position of clamps on the Clark rail 12 or the position along the leg of the lever arms may be adjusted as necessary. Continued rotation of the lever arm 36 in the clockwise direction will impose a tensile load on the ankle joint and provide an initial level of joint distraction. The initial level of distraction need only be very slight and just enough to hold the patient's leg and the apparatus firmly in position. The operating arm of each clamp is then turned to clamp the lever arms 34, 36 to establish its angular position with respect to the operating table and its axial position along the length of the leg.

With the leg and distractor held firmly in its initial position, the final desired level of joint distraction may be reached by turning the adjustment nut on the adjustment mechanism to change its position along the threaded bolt, to increase or decrease the tensile force applied to the foot strap and thus the level of distraction force. The level of distraction is generally judged by the surgeon based on his preliminary examination of the patient and considering such factors as the nature of the pathology, the age of the patient, the relative degree of ligament laxity, and other indications. After the arthroscope has been inserted into the joint, the need for increased (or even decreased) distraction may be indicated. Any desired adjustment can be easily made by turning the adjustment nut.

If additional distraction is needed beyond a level which can be adequately supported by the patient's knee only slightly flexed (FIG. I), it may be necessary to more acutely flex the knee to a position shown, for example, in FIGS. 2 and 4. The FIG. 2 angle of the lower leg may also position the posterior of the ankle joint in a more desirable position for the surgeon requiring entry through the posterior portals. The adjustment from the FIG. 1 to the FIG. 2 position is easily effected by raising the leg holder to a slightly higher position and sliding each clamp along the Clark rail toward the leg holder. These adjustments may be made simply and with little loss of time and without affecting the sterile draping.

In either of the positions shown in FIG. 1 or FIG. 2, it will be appreciated that the vertical position of the ankle above the operating table can be varied considerably and as desired by varying the position of the leg with respect to the Clark rail. Furthermore, if necessary, the leg could be flexed even more acutely than shown in the FIG. 2 position with similar adjustments to the leg holder and distractor as previously described. However, in the more acutely flexed positions, the pressure which can be applied to the popliteal area is potentially greater and circulation must be monitored.

To facilitate certain procedures or to accommodate the personal preference of the surgeon, the apparatus and method of the present invention allows further significant adjustments in the height and relative position of the ankle joint in a manner utilizing simple adjustment of the type previously described, with little loss of time, and without affecting the sterile draping. By additionally utilizing a table end which can be pivoted or tilted downwardly about a laterally disposed hinge and locked in a tilted position, the lower leg may be positioned nearly vertical or even below the level of the operating table, if desired. The hinge is positioned between the adjustable clamp for the lever arm and the clamp for the leg holder support arm. The leg holder clamp may be adjusted to position the leg holder close to the hinge to accommodate the downwardly depending positions of the leg as shown in FIGS. 3 and 4. The FIG. 3 position may be suitable for most diagnostic work and some surgery. In this position, the ankle may be placed closer vertically to the horizontal top of the operating table, but because of the downwardly dependent table end, the table does not provide as significant an obstruction to surgical procedures and access to the entire ankle joint is relatively uninhibited.

If posterior lateral entry is required or if a lower position of the ankle is more convenient to the surgeon, the table end may be tilted downwardly to an even greater acute angle with respect to the horizontal table top and locked in the FIG. 4 position. This position may require the leg holder to be positioned closer to the hinge by repositioning the leg holder clamp. This, in turn, may also necessitate repositioning the adjustable clamps on the table end to move it closer to the distal end thereof.

Should invasive distraction techniques, such as those described in prior U.S. Pat. No. 5,063,918, become necessary during procedures performed in any of the positions of FIGS. 1-4, conversion to such invasive techniques can be made quickly and without reprepping or redraping as indicated in that patent.

It is also within the scope of the present invention to utilize multiple braided sleeves that circumscribe one another or that are interwoven with one another to provide selective tensioning of the sock along the longitudinal axis of the sock 28. Those skilled in the art, in light of this disclosure, will be familiar with the points along a sock that could be advantageously constricted to attach the sock to the body part in question. Thus, by tensioning the sock longitudinally, the circumferential forces along the longitudinal axis may be adjusted, particularly where the circumference of the sock is constant, but the constricting forces are not. Moreover, interposing multiple ply sleeves may be utilized to generate a constant circumferential force along the longitudinal axis of the sock when an outer ply circumference is not constant along the longitudinal axis of the sock.

Referring to FIG. 5, it is also within the scope of the invention to incorporate a constrictor 60 at one open end of the tubular sleeve 38′ of the sock 28′, generally opposite a distal portion of the foot. In such an alternate exemplary embodiment, the constrictor 60 may comprise a string, chord, or other device used to constrict an open end of the sleeve 38′. In exemplary form, the constrictor 60 comprises a pliable chord entwined with sleeve in a circumscribing manner. Opposing ends of the constrictor 60 may be free, or one end of the constrictor fixed to the sleeve 38′, so that tension on the constrictor 60 reduces the cross-section of the sleeve 38′. Likewise, constrictors 60 may be distributed along the length of the sleeve and, in a further alternate exemplary embodiment, a constrictor 60 may be used in lieu of one or more rings 48. In this manner, tensioning forces applied to the constrictor are operative to decrease or maintain the cross-section of the sleeve.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of, any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. An orthopedic distraction sock for circumscribing at least a portion of an appendage, the distraction sock comprising:
a hollow braided sleeve having opposing open ends and including a non-braided tether mounted to the sleeve so that tension on the tether constricts the sleeve.

2. The orthopedic distraction sock of claim 1, wherein the sleeve includes a plurality of attachment loops distributed generally in parallel with a longitudinal axis of the sleeve for secondarily mounting the sleeve to a support structure.

3. The orthopedic distraction sock of claim 1, wherein the sleeve includes a plurality of attachment loops distributed along a length of the sleeve generally perpendicular to a longitudinal axis of the sleeve for secondarily mounting the sleeve to a support structure.

4. The orthopedic distraction sock of claim 1, wherein the sleeve is a bias weaved.

5. The orthopedic distraction sock of claim 1, wherein a major portion of the sleeve is substantially cylindrical.

6. The orthopedic distraction sock of claim 1, further comprising a repositionable loop mounted to the sleeve, wherein the tether is mounted to a distal open end of the sleeve and the repositionable loop is mounted to a proximal open end of the sleeve.

7. The orthopedic distraction sock of claim 1, wherein the tether comprises a plurality of lines circumferentially mounted to the sleeve approximate a distal open end of the sleeve and bound to have a substantially common point of origin.

8. The orthopedic distraction sock of claim 1, wherein the sleeve comprises a plurality of braided layers.

9. An orthopedic distraction sock for circumscribing at least a portion of an appendage, the distraction sock comprising:
a constrictable sleeve having opposing first and second open ends;
a tether mounted to the sleeve; and
a constrictor entwined with the sleeve approximate the first open end.

10. The orthopedic distraction sock of claim 9, wherein the sleeve includes a plurality of attachment loops distributed generally in parallel with a longitudinal axis of the sleeve for secondarily mounting the sleeve to a support structure.

11. The orthopedic distraction sock of claim 9, wherein the sleeve includes a plurality of attachment loops distributed along a length of the sleeve generally perpendicular to a longitudinal axis of the sleeve for secondarily mounting the sleeve to a support structure.

12. The orthopedic distraction sock of claim 9, wherein the sleeve is a bias weaved.

13. The orthopedic distraction sock of claim 9, wherein a major portion of the sleeve is substantially cylindrical.

14. The orthopedic distraction sock of claim 9, further comprising a repositionable loop mounted to the sleeve, wherein the tether is mounted to a distal open end and the repositionable loop is mounted to a proximal open end.

15. The orthopedic distraction sock of claim 9, wherein the tether comprises a plurality of lines circumferentially mounted to the sleeve approximate a distal open end and bound to have a substantially common point of origin.

16. The orthopedic distraction sock of claim 9, wherein the sleeve comprises a plurality of braided layers.

17. A method of securing the relative position of an ankle during an arthroscopic procedure, the method comprising:
retarding movement of an upper leg;
sliding a constrictable sleeve axially over at least a majority of a foot, the sleeve including a distal open end and a proximal open end nearer an ankle than the distal open end;
mounting the sleeve to a support structure; and
tensioning the sleeve by increasing a distance between the support structure and the foot to constrict the sleeve circumferentially around the foot to securely mount the sleeve to the foot, where tensioning of the sleeve, after retarding movement of the femur, is operative to maintain the relative orientation between the foot and a lower leg.

18. The method of claim 17, further comprising constricting the proximal end of the sleeve around the foot by drawing taught a constrictor of the sleeve approximate the proximal end, wherein the act of tensioning the sleeve includes tensioning the sleeve in a direction away from the ankle to constrict a more distal portion of the sleeve circumferentially around the foot to securely mount the sleeve to the foot.
